(19) United States
(12) Patent Application Publication
(10) Pub. No.: US 2008/0306355 A1
(43) Pub. Date: Dec. 11, 2008

(54) Method and System for Monitoring Gastrointestinal Function and Physiological Characteristics

(75) Inventor: Dwight Sherod Walker, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SMITHKLINE BEECHAM CORPORATION, Philadelphia, PA (US)

(21) Appl. No.: 12/117,161

(22) Filed: May 8, 2008

Related U.S. Application Data
(63) Continuation-in-part of application No. PCT/US2007/084378, filed on Nov. 12, 2007.
(60) Provisional application No. 60/866,505, filed on Nov. 20, 2006.

(51) Int. Cl. A61B 5/02; A61B 7/00; A61B 5/11

(52) U.S. Cl. 600/301; 600/586; 600/595

(57) ABSTRACT

A system and method for evaluating gastrointestinal motility and, optionally, other physiological characteristics (e.g., pulse rate) that can be effectively employed to acquire one or more signals associated with acoustic energy (i.e. sound) emanating from an abdominal region of a body and determine at least one gastrointestinal parameter or event based on the acoustic energy signal(s) is described. The gastrointestinal parameter can include a gastrointestinal event, including gastrointestinal mixing, emptying, contraction and propulsion, and gastrointestinal transit time, or a gastrointestinal system disorder, including reflux disease, irritable bowel disease, ulcerative colitis, constipation, diarrhea, and a migrating motor complex disorder.

### CROSS-REFERENCE TO RELATED APPLICATIONS

This application is a continuation-in-part of International Application No. PCT/US2007/084378, filed Nov. 12, 2007, which claims priority from U.S. Provisional Patent Application No. 60/866,505, filed Nov. 20, 2006, the contents of which is incorporated herein in its entirety.

### FIELD OF THE INVENTION

The present invention relates generally to methods for non-invasive assessment of gastrointestinal function and physiological characteristics.

### BACKGROUND OF THE INVENTION

Advances in the pharmaceutical industry in the past decades have been essential in extending the length and quality of the human life. In addition to new compounds, advances in oral medication formulation and delivery methods have helped improve efficacy while minimizing dosage and reducing side effects. However, the human body's digestive tract is heterogeneous, and differences in digestive enzymes, absorption rates, micro flora, and other factors make certain sites in the gastro-intestinal tract more or less ideal for the delivery of specific medications.

Drug companies have focused considerable efforts in targeted drug delivery, i.e. location and rate of drug delivery within the gastrointestinal (“GI”) tract. These efforts have resulted in variations in the forms of basic delivery designs, e.g., gel capsule vs. hard tablet, coating formulations, etc., and more recently, advanced control over micro- and nana-particle size. While these advances have proven beneficial, the human element remains: the GI system is intensely variable, both inter- and intra-subject. A key variable, gastrointestinal motility and, hence, gastrointestinal (or digestive) transit time, complicates determining the ideal targeted drug delivery.

Gastrointestinal motility also can, and in many instances will, have a significant impact on the clinical evaluation of the efficacy of a pharmaceutical formulation. Indeed, as is well known in the art, if an orally delivered pharmaceutical formulation, e.g., gel capsule containing a pharmaceutical formulation, exits the gastrointestinal tract prior to optimum dissolution and, hence, absorption, the efficacy of the formulation will be greatly diminished. Moreover, it has been found that in some instances, the capsule can remain in the upper gastrointestinal tract (i.e. upper fundus) for extended periods of time (e.g., >5 hrs).

It is also well known that there is a direct relationship by and between gastrointestinal motility and gastrointestinal function. Indeed, in many instances, gastrointestinal motility can reflect normal and/or abnormal gastrointestinal function, e.g., gastrointestinal obstruction.

Various methods and systems have been employed to assess gastrointestinal motility and transit time. A commonly employed method comprises gamma scintigraphy. There are, however, several significant drawbacks associated with gamma scintigraphy.

A drawback associated with gamma scintigraphy is that the method is presently limited to a small number of facilities and experts due to the issues (and controls) associated with handling radiological substances and the equipment expense. A further drawback is that large scale clinical drug trials are impractical.

Further methods and systems for assessing gastrointestinal motility include the acquisition and evaluation of gastrointestinal sounds. For example, in U.S. Pat. No. 5,301,679, a method and system are disclosed for providing diagnostic information for various diseases, including diseases of the gastrointestinal tract, by capturing body sounds with a microphone placed on the body surface or inserted orally or rectally into the gastrointestinal tract.

Other systems also employ microphones sensitive to gastrointestinal sounds within specific frequency ranges and are exemplified by Dalle, et al., “Computer Analysis in Bowel Sounds”, Computers in Biology and Medicine, Vol. 4 (3-4), pp. 247-254 (February 1975); Sugrue et al., “Computerized Phonoenterography: The Clinical Investigation of the New System”, Journal of Clinical Gastroenterology, Vol. 18, No. 2, pp. 139-144 (1994); Poynard, et al., “Qu'attendre des systemes experts pour le diagnostic des troubles fonctionnes intestinaux”, Gastroenterology Clinical Biology, pp. 45c-48c (1990).

A significant drawback associated with the conventional acoustic methods and systems is that the scope of information that can be derived from the recorded sounds is limited. Indeed, there is little, if any, disclosure directed to the relationship between gastrointestinal sounds and gastrointestinal transit times.

It would therefore be desirable to provide a method and system for evaluating gastrointestinal motility and determining gastrointestinal transit time by abdominal auscultation.

### SUMMARY OF THE INVENTION

Embodiments of the present invention provide systems and methods for monitoring gastrointestinal function and, optionally, other physiological parameters, such as pulse and respiration rates. The systems and methods of the invention can thus provide a variety of information, including gastrointestinal transit time and other physiological parameters.

In accordance with one aspect of the invention, there is provided a system and method for monitoring gastrointestinal function that can be effectively employed to acquire one or more signals associated with acoustic energy (i.e. sound) emanating from an abdominal region of a body and determine at least one gastrointestinal parameter based on the acoustic energy signal(s).

In accordance with one embodiment of the invention, there is thus provided a system for monitoring gastrointestinal function of a subject, comprising: (a) at least one sensor mountable on or in a body region of the subject, the sensor being adapted to sense acoustic energy and generate at least one acoustic energy signal representing the acoustic energy, and (b) a processing unit adapted to receive the acoustic energy signal, the processing unit being further adapted to process the acoustic energy signal and determine the occurrence of at least one gastrointestinal parameter or event.

In one embodiment, the gastrointestinal parameter comprises an event selected from the group consisting of gastrointestinal mixing, emptying, contraction and propulsion, and gastrointestinal transit time.

In one embodiment, the gastrointestinal parameter comprises an event associated with a gastrointestinal system disorder that is selected from the group consisting of reflux disease, irritable bowel disease, ulcerative colitis, constipation, diarrhea, and a mitigating motor complex disorder.

In accordance with another embodiment of the invention, there is provided a system for monitoring gastrointestinal function and physiological characteristics, comprising: (a) at least one acoustic energy sensor mountable on or in a body region of a subject, the acoustic energy sensor being adapted to sense acoustic energy representing a gastrointestinal sound generated by the subject and generate an acoustic energy signal representing the acoustic energy, (b) at least one physiological sensor mountable on or in a body region of the subject, the physiological sensor being adapted to sense a physiological characteristic associated with the subject and generate a physiological characteristic signal representing the physiological characteristic, and (c) a processing unit adapted to receive the acoustic energy and physiological characteristic signals, the processing unit being further adapted to process the acoustic energy and physiological characteristic signals and determine the occurrence of at least one gastrointestinal parameter or event as a function of the acoustic signal.

In accordance with another embodiment of the invention, there is provided a system for monitoring gastrointestinal function of a subject, comprising: (a) at least one acoustic energy sensor mountable proximate a body region of the subject, the acoustic energy sensor being adapted to sense acoustic energy generated by the subject and generate at least one acoustic energy signal representing the acoustic energy, (b) at least one spatial parameter sensor mountable proximate a body region of the subject, the spatial parameter sensor being adapted to monitor at least one spatial parameter associated with the subject's body and generate at least one spatial parameter signal representing the spatial parameter, and (c) a processing unit adapted to receive the acoustic energy and spatial parameter signals, the processing unit being further adapted to determine the occurrence of at least one gastrointestinal parameter as a function of the acoustic energy and spatial parameter signals.

In one embodiment, the spatial parameter sensor comprises a motion sensor that is adapted to monitor motion of the subject's body and the spatial parameter comprises the motion of the subject's body.

In one embodiment, the spatial parameter sensor comprises an orientation sensor that is adapted to monitor orientation of the subject's body and the spatial parameter comprises the orientation of the subject's body.

In accordance with another embodiment of the invention, there is provided a system for monitoring gastrointestinal function and physiological characteristics, comprising: (a) at least one acoustic energy sensor mountable proximate a body region of a subject, the acoustic energy sensor being adapted to sense acoustic energy generated by the subject and generate at least one acoustic energy signal representing the acoustic energy, (b) at least one spatial parameter sensor mountable proximate a body region of the subject, the spatial parameter sensor being adapted to monitor at least one spatial parameter associated with the subject's body and generate at least one spatial parameter signal representing the spatial parameter, (c) at least one physiological sensor mountable proximate a body region of the subject, the physiological sensor being adapted to sense a physiological characteristic associated with the subject and generate at least one physiological characteristic signal representing the physiological characteristic, and (d) a processing unit adapted to receive the acoustic energy, spatial parameter and physiological characteristic signals, the processing unit being further adapted to determine the occurrence of at least one gastrointestinal parameter as a function of the acoustic energy and spatial parameter signals.

In one embodiment, the spatial parameter sensor comprises a motion sensor that is adapted to monitor motion of the subject's body and the spatial parameter comprises the motion of the subject's body.

In one embodiment, the spatial parameter sensor comprises an orientation sensor that is adapted to monitor orientation of the subject's body and the spatial parameter comprises the orientation of the subject's body.

In accordance with another embodiment of the invention, there is provided a method of determining a gastrointestinal parameter associated with a subject, comprising the steps of: (a) sensing acoustic energy generated by the subject's gastrointestinal system and generating an acoustic energy signal representing the acoustic energy, (b) sensing at least one spatial parameter associated with the subject and generating a spatial parameter signal representing the spatial parameter, and (c) determining at least one gastrointestinal parameter as a function of the acoustic energy and spatial parameter signals.

In accordance with another embodiment of the invention, there is provided a method of determining a gastrointestinal parameter associated with a subject, comprising the steps of: (a) sensing acoustic energy generated by the subject's gastrointestinal system and generating an acoustic energy signal representing the acoustic energy, (b) sensing at least one spatial parameter associated with the subject and generating a spatial parameter signal representing the spatial parameter, (c) sensing a physiological characteristic associated with the subject and generate at least one physiological characteristic signal representing the physiological characteristic and (d) determining at least one gastrointestinal parameter as a function of the acoustic energy and spatial parameter signals.

In accordance with yet another embodiment of the invention, there is provided a method of monitoring gastrointestinal function and physiological characteristics of multiple subjects, comprising the steps of: (a) sensing first acoustic energy generated by a first subject's gastrointestinal system and generating a first acoustic energy signal representing said first acoustic energy, (b) sensing a first physiological characteristic associated with the first subject, (c) sensing a second physiological characteristic associated with a second subject, and (d) determining at least one gastrointestinal parameter associated with the first subject as a function of the first acoustic energy signal.

In one embodiment, the second subject comprises a fetus of the first subject.

### BRIEF DESCRIPTION OF THE DRAWINGS

FIG. 1A is an illustration of a portion of a human torso showing a typical gastrointestinal tract;

FIG. 1B is an illustration of a human stomach;

FIG. 2A is a schematic illustration of one embodiment of a gastrointestinal analysis system, according to the invention;

FIG. 2B is a schematic illustration of another embodiment of the gastrointestinal analysis system shown in FIG. 2A, according to the invention;

FIG. 3 is a further illustration of the partial human torso shown in FIG. 1, showing the placement of gastrointestinal sound (or acoustic) sensors, according to one embodiment of the invention;

FIG. 4 is a schematic illustration of an analyzer, showing the sub-systems or modules thereof, according to one embodiment of the invention;

FIG. 5 is a graphical illustration of a cumulative motion parameter (AccM) as a function of time, according to the invention;

FIG. 6 is a further illustration of a portion of a human torso having a system vest disposed thereon, according to one embodiment of the invention;

FIG. 7 is a schematic illustration of a gastrointestinal motility analysis system having additional physiological sensors, according to another embodiment of the invention;

FIG. 8 is a summary of gamma scintigraphy results acquired during a gastrointestinal motility study; and

FIGS. 9-15 are graphical illustrations of gastrointestinal sound signals, reflecting gastrointestinal sounds acquired during the gastrointestinal motility study summarized in FIG. 8.

### DETAILED DESCRIPTION OF THE INVENTION

Before describing the present invention in detail, it is to be understood that this invention is not limited to particularly exemplified structures, apparatus, systems, materials or methods as such may, of course, vary. Thus, although a number of apparatus, systems and methods similar or equivalent to those described herein can be used in the practice of the present invention, embodiments of the apparatus, systems and methods according to the present invention are described herein.

It is also to be understood that like referenced characters generally refer to the same parts or elements throughout the views shown in the figures.

Unless defined otherwise, all technical and scientific terms used herein have the same meaning as commonly understood by one having ordinary skill in the art to which the invention pertains. Further, all publications, patents and patent applications cited herein, whether supra or infra, are hereby incorporated by reference in their entirety.

Finally, as used in this specification and the appended claims, the singular forms “a”, “an”, “the” and “one” include plural referents unless the content clearly dictates otherwise. Thus, for example, reference to “a sensor” includes two or more such sensors; reference to “a gastrointestinal event” includes two or more such events and the like.

### DEFINITIONS

The term “pharmaceutical composition”, as used herein, is meant to mean and include any compound or composition of matter or combination of constituents, which, when administered to an organism (human or animal) induces a desired pharmacologic and/or physiologic effect. The term therefore encompasses substances traditionally regarded as actives, drugs, prodrugs, and bioactive agents, as well as biopharmaceuticals (e.g., peptides, hormones, nucleic acids, gene constructs, etc.).

The term “pharmaceutical”, as used herein, is meant to mean and include a pharmaceutical composition that precipitates acoustic energy or a gastrointestinal sound (or sounds) from the gastrointestinal tract when orally administered to a human or animal, such as, without limitation, pharmaceutical compositions in the form of hard tablets, gel capsules (hard and soft), caplets and other solid dosage forms.

The term “ingestible”, as used herein, is meant to mean and include any substance or item that precipitates acoustic energy or a gastrointestinal sound (or sounds) from the gastrointestinal tract when orally administered to a human or animal. An “ingestible” can thus comprise a pharmaceutical, as well as a non-pharmaceutical composition, such as, without limitation, a placebo.

The term “gastrointestinal function”, as used herein, means and includes, without limitation, the operation of all of the organs and structures associated with the gastrointestinal system.

The terms “gastrointestinal system disorder” and “adverse gastrointestinal system event”, as used herein, mean and include, without limitation, any dysfunction of the gastrointestinal system, including, without limitation, a dysfunction that impedes the digestive process, such as gastrointestinal blockage.

The term “gastrointestinal event”, as used herein means and includes an activity or function associated with the gastrointestinal system, including, without limitation, gastrointestinal mixing, emptying, contraction and propulsion. A “gastrointestinal event” can also comprise an event associated with a “gastrointestinal system disorder” or “adverse gastrointestinal system event”, such as, without limitation, reflux disease, irritable bowl disease, ulcerative colitis, constipation, diarrhea, and a mitigating motor complex (MMC) phase disorder.

The term “gastrointestinal parameter”, as used herein, means and includes a characteristic associated with gastrointestinal function, including, without limitation, a gastrointestinal event and gastrointestinal transit time.

The term “gastrointestinal sound”, as used herein, means and includes acoustic energy (and all signals embodied therein) generated by a gastrointestinal event.

The term “gastrointestinal transit time”, as used herein, is meant to mean the motile time through one or more sections of the gastrointestinal tract that can be impacted by the composition of the materials being passed, state of the gastrointestinal tract, psychological stress, gender, and other factors. “Gastrointestinal transit time” is a generic term that can be used to describe the overall gastrointestinal transit time, the fundus-rectal transit time, and various other motile times through one or more sections of the gastrointestinal tract.

The term “overall gastrointestinal transit time”, as used herein, means the motility time of a pharmaceutical or ingestible from the point it is administered via its intended route (e.g., oral, rectal) through the various sections of the gastrointestinal tract and its exit from the body.

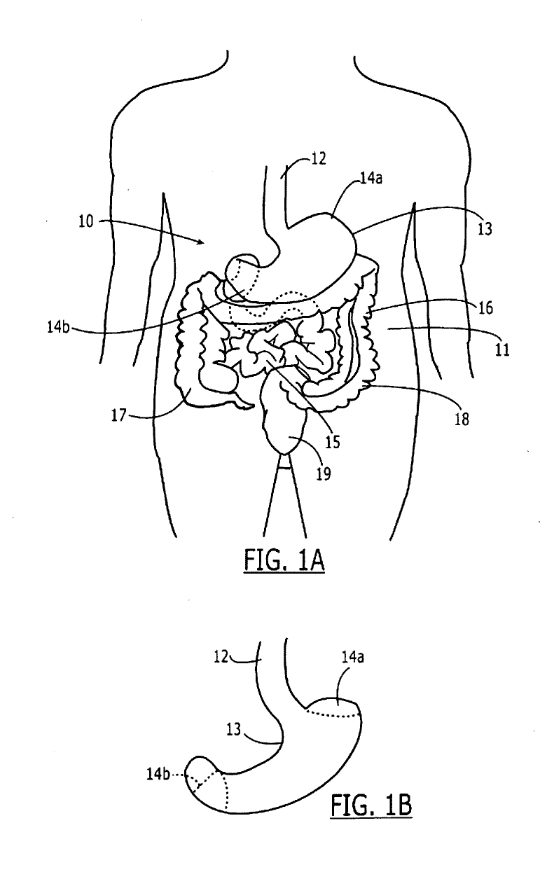

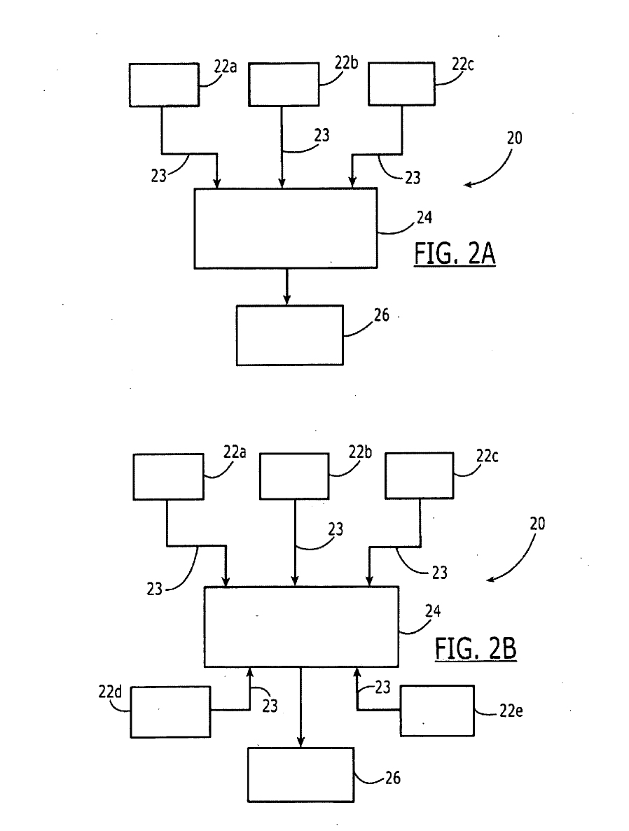

The term “fundus-rectal gastrointestinal transit time”, as used herein, means the motility time of a pharmaceutical or ingestible from entry into the fundus of the stomach through ejection from the rectum (see FIGS. 1A and 1B).

The term “signal voltage envelope”, as used herein, means an envelope that is derived from a plurality of acoustic energy signal voltages. The “signal voltage envelope” has upper and lower boundaries defined by the acoustic energy signal voltages.

The term “signal amplitude envelope”, as used herein, means an envelope that is derived from a plurality of acoustic energy signal amplitudes. The “signal amplitude envelope” has upper and lower boundaries defined by the acoustic energy signal amplitudes.

The term “V_{threshold}”, as used herein, means the minimum voltage at which values may be considered significant. According to the invention, if the signal voltage envelope is below V_{threshold}, there is no response (i.e. the signal is below the detector's sensitivity). If the signal voltage envelope is larger than V_{threshold} for longer than a pre-determined amount of time, the value is deemed significant.

The terms “physiological characteristic” and “physiological parameter”, as used herein, mean and include any characteristic associated with organism (human or animal) and/or body organ function other than a gastrointestinal parameter, including, without limitation, ECG, pulse rate, blood pressure, blood gas saturation (e.g., oxygen saturation), respiration rate skin temperature, and core temperature. The noted terms also include pharmacokinetic (PK) parameters.

The term “spatial parameter”, as used herein, means and includes any characteristic associated with a subject's body orientation (e.g., whether a subject is supine, prone, sitting, standing, etc.) and/or body motion (e.g., whether a subject is stationary, changing body position, walking, etc.).

The terms “spatial parameter value” and “spatial parameter factor”, as used herein, mean and include a numeric value representing a spatial parameter and/or the affect of a “spatial parameter” on a gastrointestinal parameter or event.

The term “subject”, as used herein, means and includes a human or an animal. The term also includes an unborn human, i.e. fetus, or animal.

The present invention provides systems and methods for monitoring gastrointestinal function and, optionally, other physiological characteristics associated with a patient or subject. As set forth in detail herein, in some embodiments, the methods and systems of the invention can be effectively employed to acquire one or more signals associated with acoustic energy (i.e. sound) emanating from an abdominal region of a subject's body and determine (i) at least one gastrointestinal parameter based on the acoustic energy signal(s) and/or the onset thereof, and/or (ii) an event associated with a gastrointestinal system disorder (and/or a gastrointestinal system disorder) and/or the onset thereof.

As discussed in detail herein, some embodiments of the systems and methods of the invention are also adapted to effectively account for spatial parameters associated with the subject, such as the subject's body orientation and/or motion.

The methods and systems of the invention can also be effectively employed to acquire one or more signals associated with a physiological parameter or characteristic, such as pulse rate, respiration rate and blood pressure.

Implementation of the methods and systems of embodiments of the present invention, as described herein, can involve performing or completing selected tasks or steps manually, automatically, or a combination thereof. In some embodiments of the present invention, several selected steps could be implemented by hardware or by software on any operating system or any firmware or a combination thereof. For example, as hardware, selected steps of embodiments of the invention could be implemented as a chip or a circuit. As software, selected steps of embodiments of the invention could be implemented as a plurality of software instructions being executed by a computer using any suitable operating system. In any case, selected steps of the method and system of the invention could be described as being performed by a data processor, such as a computing platform for executing a plurality of instructions.

Referring first to FIG. 1A, there is shown an illustration of a typical gastrointestinal tract (designated generally “10”). As illustrated in FIG. 1, the gastrointestinal tract 10 generally includes the oesophagus or esophagus 12, stomach 13, small intestines 15 and large intestines 16. The large intestines include the cecum 17, colon 18 and rectum 19.

Referring to FIG. 1B, the stomach 13 includes the fundus region (or fundus) 14a and pyloric antrum (or antrum) 14b.

As is well known in the art, gastrointestinal motility (in a normal male/female subject) is typically characterized by the repeated appearance of three distinctive phases, called the migrating motor complex (MMC). Phase 1 comprises a period or phase of no contractions. Phase 2, which follows phase 1, comprises a phase of intermittent, variable-amplitude contractions. Phase 3, which follows phase 2, comprises a phase of repetitive propagating contractions. The migrating motor complex has an average cycle of 80 to 150 min.

It is also well established and known in the art that distinctive sounds emanate from the gastrointestinal tract during each of the noted phases. See, e.g., T. Tomomasa, et al., “Gastrointestinal Sounds and Migrating Motor Complex in Fasted Humans”, The American Journal of Gastroenterology, Vol. 94, No. 2, pp. 374-381 (1999); J. Farrar, et al., “Gastrointestinal Motility as Revealed by Study of Abdominal Sounds”, Gastroenterology, Vol. 29, No. 5, pp. 789-800 (1955); W. B. Cannon, “Auscultation of the Rhythmic Sounds Produced by the Stomach and Intestines”, Laboratory of Physiology, VI, pp. 339-353 (1905).

As indicated above, although there have been various studies relating to gastrointestinal sounds and publications resulting therefrom, there is scant information relating to the relationships between gastrointestinal sounds and the migrating motor complex. There is also very little information relating to the relationship between gastrointestinal sounds and gastrointestinal transit time.

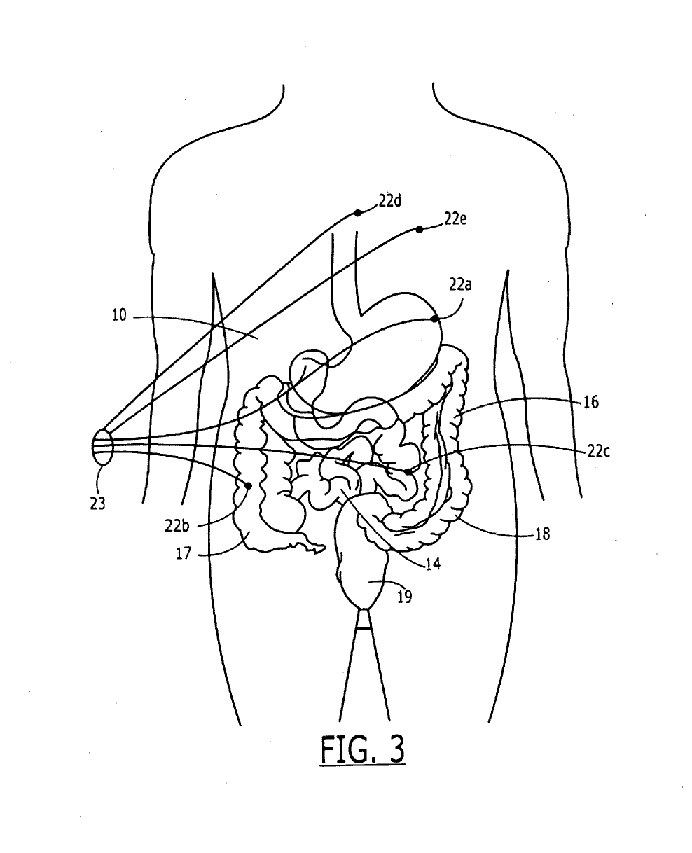

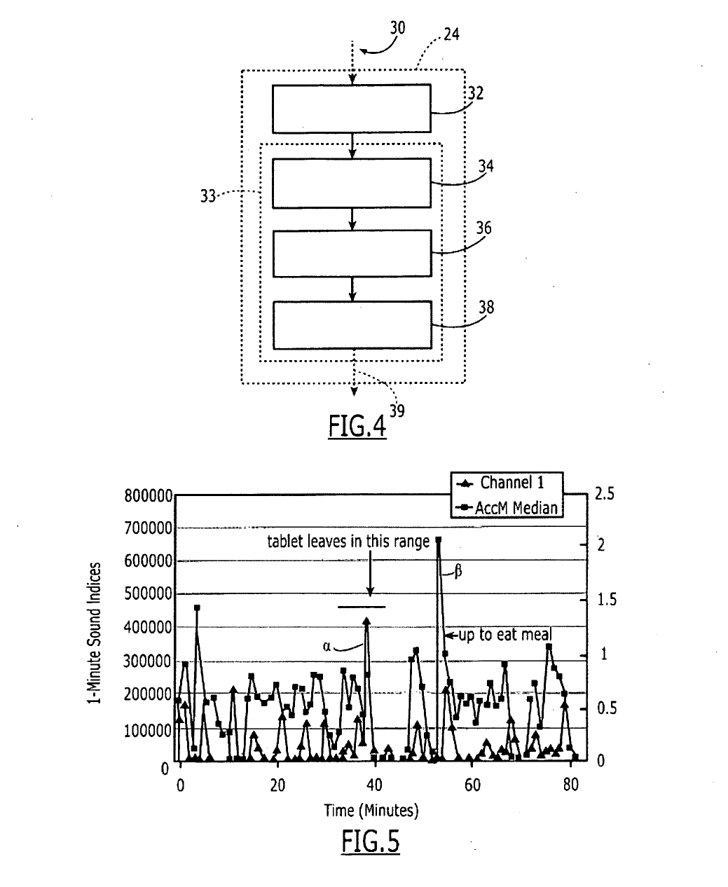

Referring now to FIG. 2A, there is shown a schematic illustration of one embodiment of a gastrointestinal analysis system 20 of the invention. As illustrated in FIG. 2A, the system 20 includes a plurality of acoustic energy sensors 22a, 22b, 22c and at least one analyzer 24. In the embodiment shown in FIG. 2A, the system 20 also includes display means 26.

According to the invention, the acoustic energy sensors 22a, 22b, 22c can independently comprise contact or non-contact transducers that detect vibrations and/or sounds at or near the skin surface of a subject and convert these vibrations and/or sounds into electrical signals. Other sensors can include internal sensors, such as intra-esophageal and intra-gastric sensors, that are introduced into the subject (or patient) using a nasal-gastric tube or the like.

By way of example only, the acoustic energy sensors 22a, 22b, 22c can be electronic stethoscopes, contact microphones, non-contact vibration sensors, such as capacitive or optical sensors, or any other suitable type of sensors. The acoustic energy sensors 22a, 22b, 22c are preferably, but not necessarily, selected to have acoustic impedance that matches the impedance of the skin surface to provide optimal acoustic coupling to the skin surface. Still further, due to background noise and the relatively low amplitude of the vibrations or sounds which are generated at or near the skin surface by gastric sounds, the acoustic energy sensors 22a, 22b, 22c are also preferably, but not necessarily, selected to provide a high signal-to-noise ratio, high sensitivity and/or good ambient noise shrouding capability.

According to the invention, the acoustic energy sensors 22a, 22b, 22c and spatial parameter sensors 22d, 22e send low level (i.e. low power) electrical signals via wires 23, or any other suitable media, such as wireless radio frequency, infrared, etc., to the analyzer 24.

A suitable acoustic energy sensor that can be employed within the scope of the invention is disclosed in U.S. Pat. No. 6,512,830.

While three acoustic energy sensors are shown in FIG. 2A, additional or fewer sensors can be used to detect gastric sounds at multiple locations on the subject's abdomen 11, or any other locations on the subject's body that are of interest and which may be useful in evaluating gastrointestinal function, and/or gastrointestinal motility (and/or transit time). For example, a single acoustic energy sensor may be strategically located on the subject's body and/or may be moved sequentially to different key locations on the subject's body to detect gastrointestinal sounds.

Referring now to FIG. 2B, there is shown another embodiment of the gastrointestinal analysis system 20. As illustrated in FIG. 2B, the system 20 similarly includes acoustic energy sensors 22a, 22b, 22c, analyzer 24 and display 26. However, in this embodiment, the system 20 further includes at least one, preferably, two spatial parameter sensors 22d, 22e.

In one embodiment of the invention, spatial parameter sensor 22d comprises a motion sensor that is adapted to monitor spatial parameters associated with the subject's body motion, e.g., whether the subject is stationary, changing body position, walking, etc., and transmit at least one motion signal representing same to the analyzer 24.

In one embodiment of the invention, spatial parameter sensor 22e comprises an orientation sensor that is adapted to monitor spatial parameters associated with the subject's body orientation, e.g., whether the subject is supine, prone, sitting, standing, etc., and transmit at least one orientation signal representing same to the analyzer 24.

As will readily be appreciated by one having ordinary skill in the art, body motion and orientation can be determined by a number of conventional methods and means, including, without limitation, optical encoders, proximity and Hall effect switches, laser interferometry and accelerometers.

As will also be appreciated by one having ordinary skill in the art, the motion and orientation sensors 22d, 22e can comprise integral, multi-function devices. Thus, in some embodiments of the invention, sensors 22d, 22e comprise multi-function 3-axis accelerometers (referred to hereinafter as “motion/orientation sensors”). By virtue of the multi-function capability of 3-axis accelerometers, in some of the noted embodiments, only one motion/orientation sensor, e.g., 2d, is employed to monitor body motion and orientation.

As discussed in detail below, the analyzer 24 can include amplifiers, filters, transient protection and other circuitry that amplifies signals sent by the acoustic energy sensors 22a, 22b, 22c, (and, optionally, motion/orientation sensors 22d, 22e) that attenuates noise signals, and/or that reduces the effects of aliasing. In particular, the analyzer 24 can include a low-pass filter having a cutoff frequency in the range of approximately 1100-1400 Hz. In one embodiment of the invention, the low-pass filter has a cutoff frequency in the range of approximately 1200-1300 Hz.

Alternatively or additionally, a high-pass filter can be incorporated within the analyzer 24. This high-pass filter may, for example, have a cutoff frequency in the range of approximately 70-90 Hz so that undesirable noise and sounds, such as muscle noise, breathing sounds, cardiac sounds, non-gastric gastrointestinal sounds or any other undesirable sounds or noise are substantially attenuated or eliminated before the signals sent by the acoustic energy sensors 22a, 22b, 22c are processed further.

The spectral energy of the most potentially corrupting non-gastrointestinal sounds is often in a frequency band of approximately 20-250 Hz. However, the amplitude of these corrupting sounds can be reduced, in some cases significantly reduced, for adult subjects by considered positioning of the acoustic energy sensors 22a, 22b, 22c.

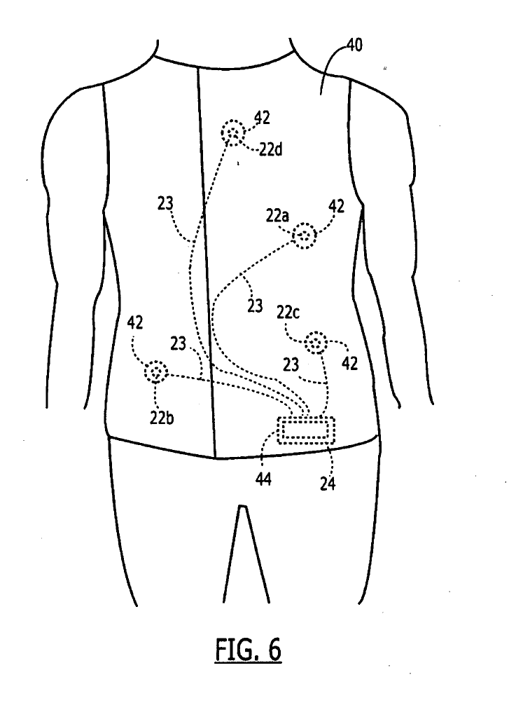

Referring now to FIG. 3, there is shown a preferred placement of acoustic energy sensors 22a, 22b, 22c, motion/orientation sensors 22d, 22e, according to one embodiment of the invention. As illustrated in FIG. 3, acoustic energy sensor 22a is preferably placed in the upper left quadrant proximate the gastric fundus, acoustic energy sensor 22b is preferably placed in the lower right quadrant proximate the cecum, and acoustic energy sensor 22c is preferably placed in the lower left quadrant proximate the small intestine, more preferably, proximate the descending colon.

According to embodiments of the invention, the acoustic energy sensors 22a, 22b, 22c can be disposed in locations other than those specifically depicted in FIG. 3 without departing from the scope and the spirit of the invention. For example, acoustic energy sensor 22a can be located on a traverse line approximately two-thirds of the distance between the umbilicus and xyphoid to the right of the midline, acoustic energy sensor 22b can be located over the left coastal margin and acoustic energy sensor 22c can be located at the midline at approximately one-half of the distance between the umbilicus and symphosis pubis.

In the embodiment illustrated in FIG. 3, the motion/orientation sensors 22d, 22e are preferably disposed proximate the anterior surface of the abdomen, preferably, proximate the center of the chest region.

According to the invention, the motion/orientation sensors 22d, 22e can similarly be disposed in locations other than those specifically depicted in FIG. 3 without departing from the scope of the invention. Further, as indicated above, only one motion/orientation sensor, such as sensor 2d, can be employed.

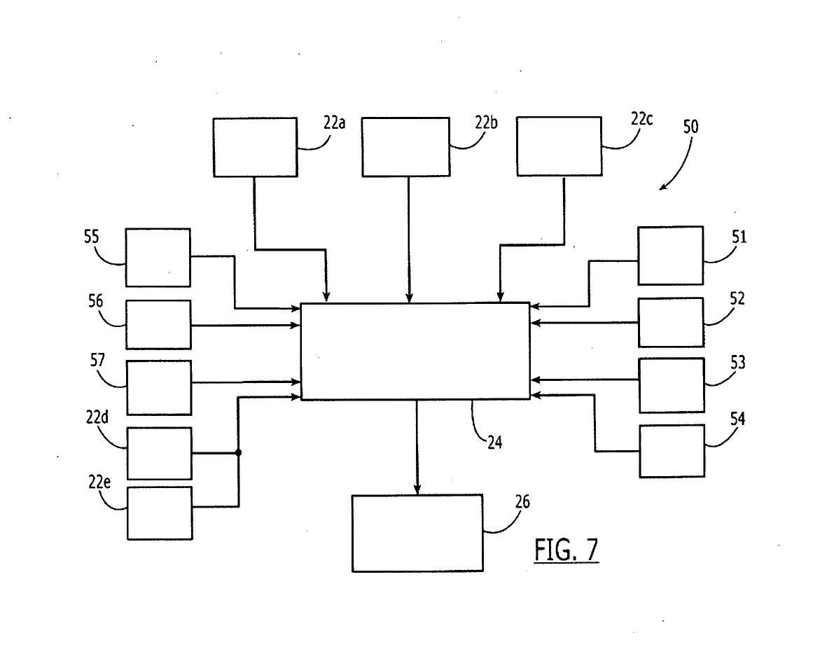

Referring to FIG. 4, according to one embodiment of the invention, the analyzer 24 is adapted to perform the following functions: (i) receive recorded acoustic energy (or gastrointestinal sound) signals from the sensors (e.g., acoustic energy sensors 22a, 22b, 22c) 30, (ii) store the acoustic energy signals in a memory medium 32, and (iii) process the acoustic energy signals (using signal processing module 33) to, according to embodiments of the invention, derive at least one gastrointestinal parameter and/or gastrointestinal event (and/or occurrence thereof) relating thereto. In some embodiments of the invention, the analyzer 24 is further adapted to compare the gastrointestinal parameter or event to at least one physiological characteristic or parameter, such as a pharmacokinetic (PK) parameter, that is induced in a subject by the administration of a pharmaceutical composition.

In some embodiments of the invention, the analyzer 24 is further adapted to determine an event associated with a gastrointestinal system disorder (and/or a gastrointestinal system disorder), such as gastrointestinal system blockage.

In another embodiment of the invention, the analyzer 24 is further adapted to (i) receive recorded motion and orientation signals from the motion/orientation sensors 22d, 22e via line 30, (ii) store the motion and/or orientation signals in the memory medium 32, and (iii) determine at least one gastrointestinal parameter and/or gastrointestinal event (and/or occurrence thereof) relating thereto and/or gastrointestinal system disorder (and/or a gastrointestinal system disorder) as a function of the recorded acoustic energy, motion and/or orientation signals. In this embodiment, the analyzer 24 thus includes algorithms and/or derived spatial parameter factors (discussed in detail below) that effectively account for the spatial parameters reflected in the motion and orientation signals in the derived gastrointestinal parameter(s), event(s) and disorder(s). For example, a spatial signal may be used to adjust an acoustic signal.

As illustrated in FIG. 4, the analyzer 24 is also adapted to provide at least one output signal 39 representing recorded acoustic energy and/or the subject's body motion and/or orientation and/or, according to further envisioned embodiments of the invention (discussed below), a physiological characteristic.

According to embodiments of the invention, the signal processing module 33 is adapted to also perform the following: (i) filter extraneous artifacts from the signals 34, (ii) determine a signal amplitude envelope based on the signals 36, and (iii) determine the dominant frequency of the signals 38.

According to the invention, the filtering step 34 can be performed with software, e.g., computer program, or hardware. Thus, in some embodiments of the invention, the analyzer 24 is programmed to filter the acoustic energy signals and extract the frequency band of interest from the signals.

In one embodiment, the frequency of interest is in the range of approximately 70-1400 Hz. In another embodiment, the frequency of interest is in the range of approximately 90-1200 Hz.

According to the invention, various conventional programs can be employed within the scope of the invention to perform the noted filtering step 34.

In other embodiments of the invention, the filtering step 34 is performed via hardware. In one embodiment, the analyzer circuit includes high and low pass filters that are adapted to filter the extraneous artifacts from the signals 34. According to the invention, various high and low pass filters can be employed within the scope of the invention. In one embodiment, the high pass filter comprises a Blackman windowed, balanced 401-tap FIR with a cutoff set at 80 Hz and the low pass filter comprises a Blackman windowed, balanced 400-tap FIR with a cutoff set at 1250 Hz.

In one embodiment, the signal amplitude envelope is determined using a sliding Hilbert transform with a 5 μsec window. As is well known in the art, Hilbert transforms are commonly used to determine a signal envelope. See, e.g., Tomomasa, et al., “Gastrointestinal Sounds and Migrating Motor Complex in Fasted Humans”, The American Journal of Gastroenterology, Vol. 94, No. 2, pp. 374-381 (1999); Farrar, et al., “Gastrointestinal Motility as Revealed by Study of Abdominal Sounds”, Gastroenterology, Vol. 29, No. 5, pp. 789-800 (1955); which are incorporated by reference herein.

Applicants have found that the Hilbert transform smoothed out the short “pops”, i.e. intermittent acoustic energy spikes, and transformed the bipolar acoustic energy signals into a signal that can be readily analyzed using a simple V_{threshold}, as defined above.

According to embodiments of the invention, the dominant frequency of the acoustic energy signals can similarly be determined by various conventional means. In one embodiment, the dominant frequency was determined by isolating peaks>V_{threshold} for time>5 μsec.

As indicated above, a key feature and advantage of the embodiments of the present invention is the ability of the gastrointestinal analysis systems and methods to effectively account for spatial parameters, i.e. body motion and orientation, in determinations of gastrointestinal parameters, events and disorders.

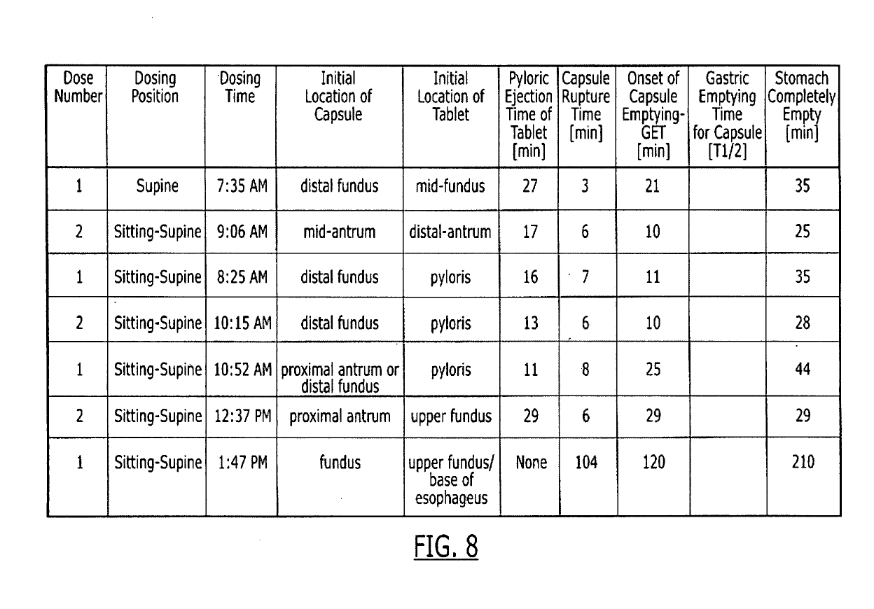

Referring to FIG. 5, there is shown a graphical illustration of a cumulative composite motion measure (AccM) as a function of time. The AccM is a summation of both the X and Y body axis. As demonstrated in FIG. 5, the acoustic signal of the acoustic sensor (Channel 1) captures the tablet leaving the stomach (denoted “α”) while the motion/orientation signal of the motion/orientation sensor (i.e. 3-axis accelerometer) captures the subject rising to an upright position to eat a meal (denoted “β”). FIG. 5 further demonstrates a shift (i.e. increase) in the recorded signal resulting from the subject's motion.

Accordingly, in some embodiments of the invention, the analyzer 24 includes algorithms and/or derived spatial parameter factors that effectively account for the spatial parameters reflected in the motion and orientation signals in the derived gastrointestinal parameter(s), event(s) and disorder(s).

As stated above, spatial parameters, i.e. body motion and orientation, can be determined by a number of conventional means, such as optical encoders, proximity and Hall effect switches, laser interferometers and multi-axis accelerometers. According to some embodiments of the invention, the output of these predominantly digital devices, i.e. motion and/or orientation signals, is translated into a spatial parameter value or factor.

A subject matrix is then generated and stored in the memory medium 32; the matrix including a plurality of body positions and motions, and corresponding spatial parameter factors.

Referring to Table I, there is shown an exemplar subject matrix. As shown in Table I, when the subject is standing and still the maximum value or spatial parameter is “0 1 1”, as reflected in the X, Y and Z axis outputs.

TABLE I

| Body position | X axis output (v) | Y axis output (v) | Z axis output (v) |
|---|---|---|---|
| Supine left | 0 | 0 | 0 |
| Supine right | 1 | 0 | 0 |
| Standing still | 0 | 1 | 1 |
| Sitting still | 0 | 0.5 | 0 |
| Walking | 0-1 | 1 | 0-1 |

The spatial parameter factor can then be employed to adjust the recorded acoustic signal. For example, one could adjust the V_{threshold} for each of the acoustic energy sensors (e.g., 22a, 22b, 22c) based on predicted GI activity at that location.

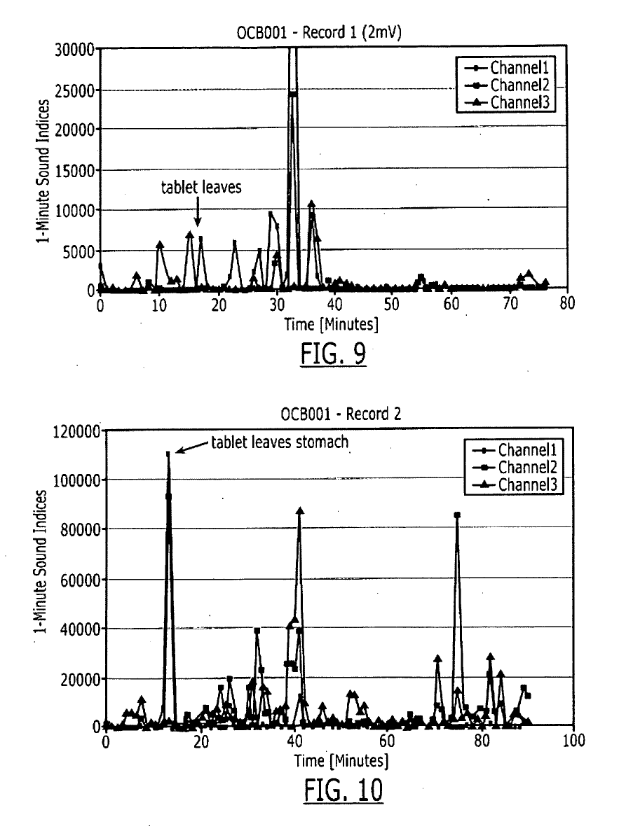

To take a particular spatial example, if the spatial factor is one (0 1-1, i.e. standing), one could place a greater emphasis on the acoustic signals of acoustic energy sensors 22b and 22c (see FIG. 6) in determining GI movement, as these sensors would be more proximate to the internal source of the acoustic signal.

A further example would be in determining orientation during sleeping, which is taught to relate to gastric emptying. If, during sleep, the spatial parameter factor was (1 0 0), one would expect enhanced gastric emptying due to the stomach contents pooling of the pylorus. In this example, one could place a greater emphasis on the acoustic signals of acoustic energy sensors 22a and 22c (see FIG. 6).

Referring back to FIGS. 2A and 2B, according to the invention, the display means 26 can comprise any suitable medium that is capable of providing at least one visual display representing recorded acoustic energy signals (pre- and post-processed) and/or body motion and/or body orientation and/or recorded physiological characteristics. In one embodiment, the display means 26 comprises a computer monitor.

According to other embodiments of the invention, the display means 26 can also comprise an audible display. The audible display can be adapted to provide a sound or tone representing, for example, body motion, a gastrointestinal event or a MMC phase. The audible display can be further adapted to provide different sounds or tones representing body motion or a selective gastrointestinal event or a gastrointestinal system disorder or MMC phases or characteristic relating thereto, e.g., initiation of a phase.

The display means 26 can also provide at least one visual display representing recorded acoustic energy signals (pre- and post-processed) and/or body motion and/or body orientation and/or recorded physiological characteristics, and at least one audible sound or tone representing body motion or at least one gastrointestinal event or physiological characteristic.

As will be appreciated by one having ordinary skill in the art, the display means 26 can also be an integral component or feature of the analyzer 24.

As will be appreciated by one having ordinary skill in the art, the acoustic energy sensors 22a, 22b, 22c, motion sensor 22d, and orientation sensor 22e (or multi-function motion/orientation sensors 22d, 22e) of the invention can be positioned on a subject's body in various conventional means. By way of example, the sensors 22a, 22b, 22c, 22d, 22e can include an adhesive ring or surface on the housing that is adapted to temporarily engage the skin of the subject. The sensors 22a, 22b, 22c, 22d, 22e can also be attached to the subject's skin via a strip of medical tape or elastic bandage.

Referring now to FIG. 6, in one embodiment of the invention, the acoustic energy sensors 22a, 22b, 22c, and a motion/orientation sensor 22d are positioned and maintained in a substantially static position against the subject's body via a vest 40. According to the invention, the vest 40 can comprise various sizes and materials.

In one embodiment, the vest 40 is adjustable and comprises a light weight, mesh material, e.g., nylon or Lycra®. In one embodiment of the invention, the vest 40 includes at least one pocket that is configured to receive and seat at least one sensor. Preferably, the vest 40 includes a plurality of pockets that are configured to receive and position a plurality of sensors; the pockets being positioned to correspond to selective positions on a subject's body when worn by the subject.

In another envisioned embodiment, the vest 40 and sensor(s) include a simple male-female snap system. In one embodiment, the vest 40 can include a plurality of positioned female portions of the snap system and the sensors can include a male portion that can engage and, hence, be secured on the vest 40 by the receiving vest female portions. In other embodiments, the vest 40 can include a plurality of positioned male portions of the snap system and the sensors can include a female portion that can engage and, hence, be secured on the vest 40 by the receiving vest male portions.

In some embodiments of the invention, the vest 40 includes at least one pocket that is adapted to receive and seat an acoustic energy sensor, e.g., sensor 22a. The vest 40 also preferably includes an analyzer pocket that is adapted to receive and seat the analyzer 24.

In the embodiment shown in FIG. 6, the vest 40 includes at least four (4) pockets 42 adapted to receive and seat acoustic energy sensors 22a, 22b, 22c, and the motion/orientation sensor 22d. The vest 40 also includes an analyzer pocket 44 that is adapted to receive the analyzer 24.

As will be readily apparent to one having ordinary skill in the art, the vest 40 provides the system 20 with mobility.

In further envisioned embodiments of the invention, the gastrointestinal motility analysis system 20 includes at least one, preferably, a plurality of additional sensors, i.e. physiological sensors, which are adapted to record one or more physiological characteristics. Such physiological characteristics include, without limitation, ECG, pulse rate, SO₂, skin temperature, core temperature and respiration rate.

According to embodiments of the invention, the additional physiological sensors can be strategically positioned on a subject to monitor and/or evaluate one or more physiological characteristics. By way of example, a first physiological sensor (i.e. pulse rate sensor) can be disposed proximate the subject's heart to monitor pulse rate and a second physiological sensor (i.e. respiration rate sensor) can be disposed proximate a diaphragm to monitor the subject's respiration rate.

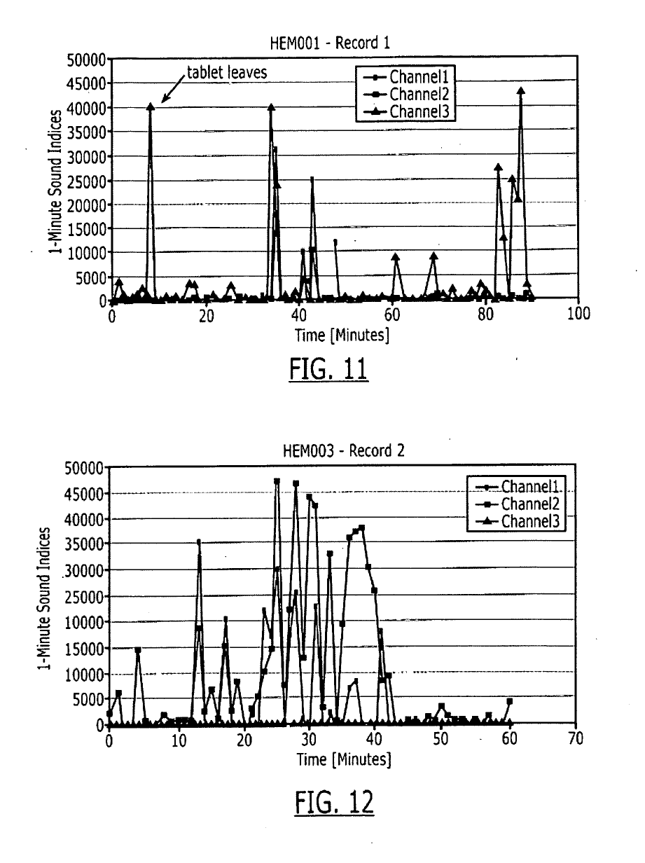

Referring now to FIG. 7, there is shown a schematic illustration of one embodiment of a gastrointestinal motility analysis system 50, according to the present invention. As illustrated in FIG. 7, the system 50 includes multiple function sensors 22a-22e and 51-58 for monitoring gastrointestinal function (or motility), body motion and orientation, and physiological characteristics of a subject.

In one embodiment of the invention, physiological sensor 51 comprises an ECG sensor adapted to monitor cardiac performance and/or function, physiological sensor 52 comprises a pulse rate sensor adapted to monitor the subject's pulse rate, physiological sensor 53 comprises an SO₂ sensor adapted to monitor the subject's blood oxygen level, physiological sensor 54 comprises a first temperature sensor adapted to monitor the subject's skin temperature, physiological sensor 55 comprises a second temperature sensor adapted to monitor the subject's core temperature, and physiological sensor 56 comprises a respiration sensor that is adapted to monitor the subject's respiration rate and tidal volume.

As illustrated in FIG. 7, the system 50 also includes one additional sensor 57. In one embodiment, sensor 57 comprises an acoustic sensor that is adapted to monitor non-gastrointestinal related acoustic energy, such as a cough. According to the invention, the signals from the acoustic sensor 57 can be used to identify and extract non-gastrointestinal related signals or artifacts that may have been recorded by the acoustic energy sensors 22a, 22b, 22c.

According to the invention, the additional sensors 51-57 can similarly be attached directly to the skin of the subject. The sensors 51-57 can also be incorporated into vest 40, as described above.

As indicated above, while the system 50 is shown with three acoustic energy sensors 22a, 22b, 22c, the system 50 can include less than three acoustic energy sensors, e.g., sensor 22a, or more such sensors.

It is also to be understood that while the system 50 is shown with twelve (12) sensors, i.e. sensors 22a-22e and 51-57, the system 50 can include any number of the sensors, e.g. one sensor, three sensor, six sensors, etc., and/or any combination of at least one of the acoustic energy sensors 22a-22c and zero or more of the sensors 51-57. For example, the system 50 can include acoustic energy sensors 22a, 22b, motion/orientation sensor 22d, and physiological sensors 52 and 57 or acoustic energy sensor 22a and physiological sensors 52 and 56.

Gastrointestinal systems, according to embodiments of the invention, including system 50, can also be effectively employed to monitor gastrointestinal function and physiological characteristics of multiple subjects. By way of example, in the case of a pregnant subject, three or more sensors can be strategically positioned on the pregnant subject's body to monitor gastrointestinal function and at least one physiological characteristic of the pregnant subject and at least one physiological characteristic of the unborn child, e.g., a gastrointestinal sensor (e.g. acoustic energy sensor 22a) disposed proximate the pregnant subject's abdominal region to monitor gastrointestinal motility, a first pulse rate sensor (e.g., physiological sensor 52) disposed proximate the pregnant subject's heart to monitor the pregnant subject's pulse rate, and a second pulse rate sensor disposed proximate the pregnant subject's abdominal region (and, hence, unborn child) to monitor the unborn child's pulse rate.

Method and system embodiments of the present invention can thus be effectively employed in numerous applications. The applications include, without limitation, the following:
To monitor gastrointestinal motility during research of a pharmaceutical composition, and clinical trials related thereto, to better assess research and clinical data.
To monitor gastrointestinal function and/or motility and determine abnormalities, i.e. gastrointestinal system disorders, associated therewith.
To monitor gastrointestinal function and/or motility during pregnancy; particularly, high risk pregnancies where gastrointestinal obstruction is often encountered.
To monitor gastrointestinal function and/or motility of a pregnant subject and physiological characteristics of the pregnant subject and unborn child, e.g., pulse rate, during pregnancy.

The methods and systems of the present invention can also be readily employed to facilitate the diagnosis and treatment of various eating disorders. Indeed, as is well known in the art, various gastrointestinal events and, hence, the acoustic energy (or sound) associated therewith, reflect digestive activity (or the lack thereof). By way of example, an extended period of time (e.g., 12 hours) without one or more phases of a migrating motor complex (MMC) could be indicative of a bulimic or anorexic subject. Conversely, an extended period of repeated MMC phases could be indicative of excessive overeating.

### EXAMPLES

The following examples are provided to enable those skilled in the art to more clearly understand and practice the present invention. They should not be considered as limiting the scope of the invention, but merely as being illustrated as representative thereof.

### Example 1

Six male subjects were initially provided with a health assessment. All subjects passed the initial health assessment. The subjects then spent the night at the test center and fasted (i.e. water only) for at least 11 hours before the study began.

Three acoustic energy sensors were positioned on each subject. Sensor #1 was placed 4-6 inches below the patient's right nipple, i.e. proximate the gastric fundus. Sensor #2 was placed 11-11.5 inches below the right nipple, i.e. proximate the cecum. Sensor #3 was placed in the Lower Left Quadrant, approximately 11 inches below the left nipple, i.e. proximate the loudest part of the small intestine/descending colon. The sensors were held firmly against each subject's body by a lightweight, close fitting nylon mesh vest, such as vest 40.

The sensors were custom modified Welch-Allen Master Elite Plus Stethoscopes. Unlike traditional stethoscopes, these pressure-based microphones employ a technology that is less sensitive to indirect vibration and hence ambient noise. Further, the sensors also contain signal processing circuitry that improves signal-to-noise ratio and deliver either traditional audio, or mono line-out signals.

Without disturbing the microphone head or signal processing electronics, the housing was removed and the microphones were repackaged passing the power and line-out signals to custom front-end analog electronics with long wiring that allows for patient mobility. Additionally, the volume was set at maximum and the onboard filtering was set to “all-pass”, which encompasses a frequency band in the range of 100-1200 Hz.

All microphone channels were amplified and low-pass filtered via an analog 2-pole 1200 Hz low-pass Bessel filter, and then sampled onto a National Instruments DAQPad-6015 at 8000 Hz. Data was recorded in 10-minute segments and post processed via software written in National Instruments LabVIEW 7.1.

Gamma scintigraphy was also performed simultaneously to assess gastrointestinal motility. A dissolvable hard gelatin capsule and a non-disintegrating tablet with radioactive markers (¹¹¹InCl₃ and ^{99m}Tc-DTPA, respectively) were administered to each subject. The tablet and the capsule were taken simultaneously with a glass of water, since it is known that capsules taken without water can stick to the esophagus for up to two hours.

As is well known in the art, the radionucleotide markers emit gamma rays of different characteristic energies. Thus, the tablet and capsule's contents could be separately tracked.

Removable stickers containing small point sources of ¹¹¹InCl₃ encased in plastic were placed on the chest and hip of each subject as a reference to ensure consistent placement of the subject under the gamma camera in between pictures. Pictures were taken every 20 seconds and integrated images stored every 1 minute by the gamma scintigraphy system. Tablet and capsule position in the gastrointestinal tract were determined and recorded for subsequent analysis.

Gastrointestinal sounds were also recorded during the ingestion of the dissolvable hard gelatin capsule and a non-disintegrating tablet. The recorded sounds i.e. sound files were stored in an analyzer according to embodiments of the invention. The sound files were processed as discussed above.

During the first of the study, the subjects were asked to remain quiet in a supine position under the gamma scintigraphy camera. Several parameters were subsequently analyzed. Individual sound dominant frequency, duration, and intensity were also all calculated.

The Sound Index (or SI) was also calculated. SI, as used herein, means the sum of the absolute amplitudes for all detected sound over a one (1) minute time period, expressed as mV/min.

As discussed above, it is known that in a fasted state, upper tract digestion occurs in a 4-stage cyclic pattern with the largest contractions of the stomach (i.e., Phase 3) usually initiated with a migrating motor complex (MMC) that proceeds from the stomach towards the ileum of the small intestine. The period between MMC's has been well established and is typically around 2 hours (although times ranging from 1 to 3 hours are not uncommon).

During the studies, clearly identifiable MMC's were observed in most subjects (˜66.7%) as identified by large SI's in all three sensors; with Sensors #1 and #2 being the loudest.

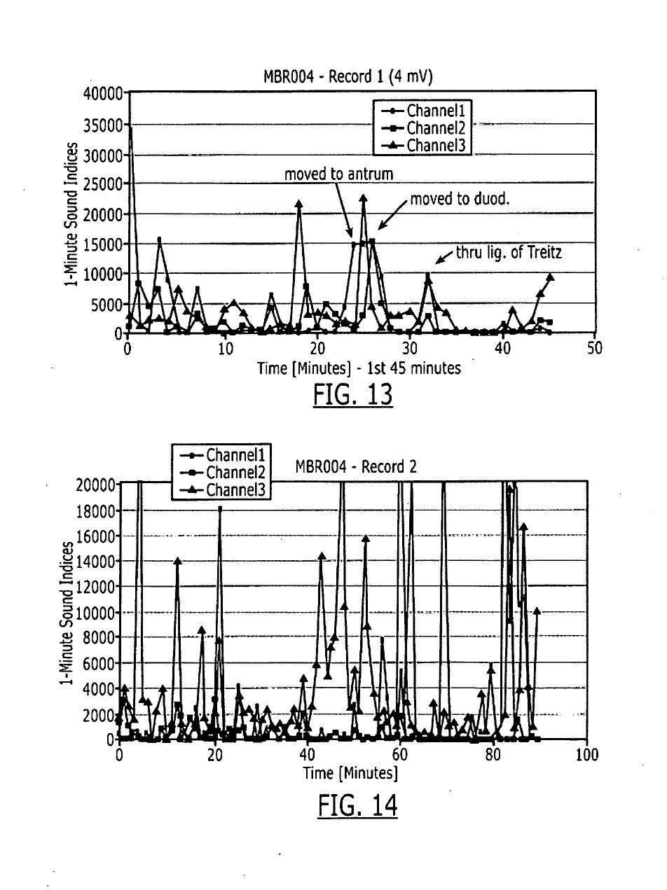

Referring now to FIG. 8, there is shown a summary of the gamma scintigraphy assessment. As reflected in FIG. 8, in all studies, but one, i.e. an “outlier”, gamma scintigraphy determined that the tablets were ejected from the stomach between 11-29 minutes (mean 18.88 min). Thus, it can be inferred that the test tablets were passed with the liquid from the stomach.

Interestingly, the “outlier” displayed an MMC without tablet movement. Tablet movement within the stomach only came later corresponding to a large sound and SI in channel 1 around 1 hour, 40 minutes. Complete tablet ejection did not occur during the entire duration of the study, i.e. 5 hours and 51 minutes. The cause of this is uncertain, but highlights the need for gastrointestinal transit monitoring.

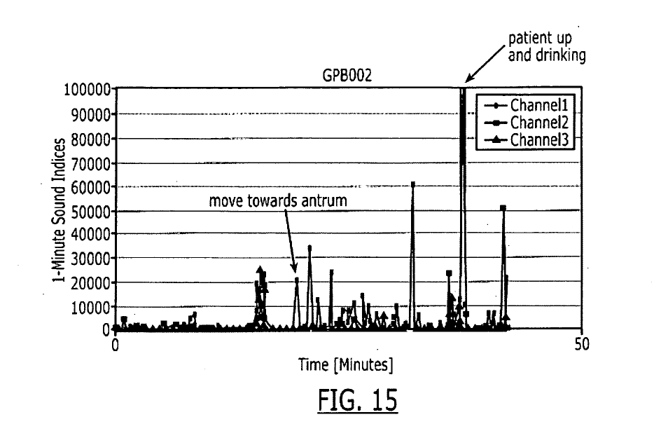

Referring now to FIGS. 9-15, there are shown graphs reflecting the sounds recorded by the sensors, i.e. minute sound indices versus time. As reflected in FIGS. 9-15, in all 6 studies, where gastric emptying of the tablet did occur during monitoring, significant bowel sounds and SI's were recorded at the time of emptying. In 5 subjects, channel #1 (or Sensor #1), which monitored gastric sounds, produced the highest SI recorded to that point.

Tablets that landed in the antrum, where muscular activity occurs, moved corresponding to first large sound in channel #1. Tablets that landed in the antrum generally took two or three large SI's to move, with the first or second SI corresponding to movement into the fundus.

For the “outlier”, bowel sounds generally matched the scintigraphy data. There appeared to be an MMC around 1 hour, 40 minutes (i.e. strong signal in all three channels), which did not affect the tablet's movement. However, afterwards, the next considerable SI was incident with movement of the tablet from the initial location of upper fundus to the antrum of the stomach. Afterwards, there were intermittent sounds recorded in channel 1, but no tablet movement. Notable though, are the very low levels of sound in the other two sensors, implying overall gastrointestinal quiescence.

The results of this study reflect that in subjects that are quiet in a supine position, discernable bowel sounds recorded by a sensor of the invention correspond to tablet ejection, as shown by gamma scintigraphy. Indeed, movements of tablet position (antrum or fundus) in the stomach were also marked by large sounds. Overall quiet sounds were detected in the patient who never experienced gastric tablet ejection. MMC's were also clearly identifiable in several subjects.

As will be readily apparent to one skilled in the art, embodiments of the present invention can provide one or more advantages, such as:
The provision of a method and system for monitoring gastrointestinal motility that can be effectively employed during research of a pharmaceutical composition, and clinical trials related thereto, to better assess research and clinical data.
The provision of a method and system for monitoring gastrointestinal motility that has the potential to reduce the time and resources associated with research of a pharmaceutical composition and clinical trials related thereto.
The provision of a method and system for monitoring gastrointestinal function that can be readily employed by a medical practitioner as a diagnostic aid during assessments of gastrointestinal behavior.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A system for monitoring gastrointestinal function of a subject, comprising:
at least one sensor mountable proximate a body region of the subject, said sensor being adapted to sense acoustic energy and generate at least one acoustic energy signal representing said acoustic energy; and
a processing unit adapted to receive said acoustic energy signal, said processing unit being further adapted to process said acoustic energy signal and determine the occurrence of at least one gastrointestinal parameter therefrom.

2. The system of claim 1, wherein said gastrointestinal parameter comprises an event selected from the group consisting of gastrointestinal mixing, emptying, contraction and propulsion, and gastrointestinal transit time.

3. The system of claim 1, wherein said gastrointestinal parameter comprises an event associated with a gastrointestinal system disorder, said gastrointestinal system disorder being selected from the group consisting of reflux disease, irritable bowel disease, ulcerative colitis, constipation, diarrhea, and a mitigating motor complex disorder.

4. The system of claim 1, wherein said sensor generates a plurality of acoustic energy signals representing said acoustic energy.

5. The system of claim 4, wherein said processing unit is adapted to receive and process said plurality of acoustic energy signals and determine the occurrence of at least a first gastrointestinal parameter therefrom.

6. The system of claim 5, wherein said first gastrointestinal parameter comprises gastrointestinal transit time.

7. A system for monitoring gastrointestinal function of a subject, comprising:
at least one acoustic energy sensor mountable proximate a body region of the subject, said acoustic energy sensor being adapted to sense acoustic energy generated by the subject and generate at least one acoustic energy signal representing said acoustic energy;
at least one spatial parameter sensor mountable proximate a body region of the subject, said spatial parameter sensor being adapted to monitor at least one spatial parameter associated with the subject's body and generate at least one spatial parameter signal representing said spatial parameter; and
a processing unit adapted to receive said acoustic energy and spatial parameter signals, said processing unit being further adapted to determine the occurrence of at least one gastrointestinal parameter as a function of said acoustic energy and spatial parameter signals.

8. The system of claim 7, wherein said spatial parameter sensor comprises a motion sensor that is adapted to monitor motion of the subject's body.

9. The system of claim 8, wherein said spatial parameter comprises said motion of the subject's body.

10. The system of claim 7, wherein said spatial parameter sensor comprises an orientation sensor that is adapted to monitor orientation of the subject's body.

11. The system of claim 10, wherein said spatial parameter comprises said orientation of the subject's body.

12. The system of claim 7, wherein said gastrointestinal parameter comprises an event selected from the group consisting of gastrointestinal mixing, emptying, contraction and propulsion, and gastrointestinal transit time.

13. The system of claim 7, wherein said gastrointestinal parameter comprises an event associated with a gastrointestinal system disorder, said gastrointestinal system disorder being selected from the group consisting of reflux disease, irritable bowel disease, ulcerative colitis, constipation, diarrhea, and a mitigating motor complex disorder.

14. A system for monitoring gastrointestinal function and physiological characteristics, comprising:
at least one acoustic energy sensor mountable proximate a body region of a subject, said acoustic energy sensor being adapted to sense acoustic energy representing a gastrointestinal sound generated by said subject and generate at least a first acoustic energy signal representing said acoustic energy;
at least one physiological sensor mountable proximate a body region of said subject, said physiological sensor being adapted to sense a physiological characteristic associated with said subject and generate at least a first physiological characteristic signal representing said physiological characteristic; and
a processing unit adapted to receive said first acoustic energy and physiological characteristic signals, said processing unit being further adapted to process said first acoustic energy signal and determine the occurrence of at least one gastrointestinal parameter therefrom.

15. The system of claim 14, wherein said gastrointestinal parameter comprises an event selected from the group consisting of gastrointestinal mixing, emptying, contraction and propulsion, and gastrointestinal transit time.

16. The system of claim 14, wherein said gastrointestinal parameter comprises an event associated with a gastrointestinal system disorder, said gastrointestinal system disorder being selected from the group consisting of reflux disease, irritable bowel disease, ulcerative colitis, constipation, diarrhea, and a mitigating motor complex disorder.

17. The system of claim 14, wherein said physiological characteristic comprises a physiological characteristic selected from the group consisting of pulse rate, blood pressure, blood gas saturation, respiration rate, skin temperature, and electrical impulses associated with heart function.

18. A system for monitoring gastrointestinal function and physiological characteristics, comprising:
at least one acoustic energy sensor mountable proximate a body region of a subject, said acoustic energy sensor being adapted to sense acoustic energy representing a gastrointestinal sound generated by said subject and generate at least one acoustic energy signal representing said acoustic energy;
at least one spatial parameter sensor mountable proximate a body region of the subject, said spatial parameter sensor being adapted to monitor at least one spatial parameter associated with the subject's body and generate at least one spatial parameter signal representing said spatial parameter;
at least one physiological sensor mountable proximate a body region of said subject, said physiological sensor being adapted to sense a physiological characteristic associated with said subject and generate at least one physiological characteristic signal representing said physiological characteristic; and
a processing unit adapted to receive said acoustic energy, spatial parameter and physiological characteristic signals, said processing unit being further adapted to determine the occurrence of at least one gastrointestinal parameter as a function of said acoustic energy and spatial parameter signals.

19. The system of claim 18, wherein said spatial parameter sensor comprises a motion sensor that is adapted to monitor motion of the subject's body.

20. The system of claim 19, wherein said spatial parameter comprises said motion of the subject's body.

21. The system of claim 18, wherein said spatial parameter sensor comprises an orientation sensor that is adapted to monitor orientation of the subject's body.

22. The system of claim 21, wherein said spatial parameter comprises said orientation of the subject's body.

23. The system of claim 18, wherein said gastrointestinal parameter comprises an event selected from the group consisting of gastrointestinal mixing, emptying, contraction and propulsion, and gastrointestinal transit time.

24. The system of claim 18, wherein said gastrointestinal parameter comprises an event associated with a gastrointestinal system disorder, said gastrointestinal system disorder being selected from the group consisting of reflux disease, irritable bowel disease, ulcerative colitis, constipation, diarrhea, and a mitigating motor complex disorder.

25. The system of claim 18, wherein said physiological characteristic comprises a physiological characteristic selected from the group consisting of pulse rate, blood pressure, blood gas saturation, respiration rate, skin temperature, and electrical impulses associated with heart function.

26. A method of determining a gastrointestinal parameter associated with a subject, comprising the steps of:
sensing acoustic energy generated by the subject's gastrointestinal system and generating an acoustic energy signal representing said acoustic energy;
sensing at least one spatial parameter associated with the subject and generating a spatial parameter signal representing said spatial parameter; and
determining at least one gastrointestinal parameter as a function of said acoustic energy and spatial parameter signals.

27. The method of claim 26, further comprising utilizing said spatial parameter signal to adjust said sensing of acoustic energy.

28. The method of claim 26, wherein said spatial parameter comprises said motion of the subject's body.

29. The method of claim 26, wherein said spatial parameter comprises said orientation of the subject's body.

30. The system of claim 26, wherein said gastrointestinal parameter comprises an event selected from the group consisting of gastrointestinal mixing, emptying, contraction and propulsion, and gastrointestinal transit time.

31. The system of claim 26, wherein said gastrointestinal parameter comprises an event associated with a gastrointestinal system disorder, said gastrointestinal system disorder being selected from the group consisting of reflux disease, irritable bowel disease, ulcerative colitis, constipation, diarrhea, and a mitigating motor complex disorder.

32. A method of determining a gastrointestinal parameter associated with a subject, comprising the steps of:
sensing acoustic energy generated by the subject's gastrointestinal system and generating an acoustic energy signal representing said acoustic energy;
sensing at least one spatial parameter associated with the subject and generating a spatial parameter signal representing said spatial parameter;
sensing at least one physiological characteristic associated with the subject and generating at least one physiological characteristic signal representing said physiological characteristic; and
determining at least one gastrointestinal parameter as a function of said acoustic energy and spatial parameter signals.

33. The method of claim 32, further comprising utilizing said spatial parameter signal to adjust said sensing of acoustic energy.

34. The method of claim 32, wherein said spatial parameter comprises said motion of the subject's body.

35. The method of claim 32, wherein said spatial parameter comprises said orientation of the subject's body.

36. The method of claim 32, wherein said gastrointestinal parameter comprises an event selected from the group consisting of gastrointestinal mixing, emptying, contraction and propulsion, and gastrointestinal transit time.

37. The method of claim 32, wherein said gastrointestinal parameter comprises an event associated with a gastrointestinal system disorder, said gastrointestinal system disorder being selected from the group consisting of reflux disease, irritable bowel disease, ulcerative colitis, constipation, diarrhea, and a mitigating motor complex disorder.

38. The method of claim 32, wherein said physiological characteristic comprises a physiological characteristic selected from the group consisting of pulse rate, blood pressure, blood gas saturation, respiration rate, skin temperature, and electrical impulses associated with heart function.

39. A method of monitoring gastrointestinal function and physiological characteristics of multiple subjects, comprising the steps of:
sensing first acoustic energy generated by a first subject's gastrointestinal system and generating a first acoustic energy signal representing said first acoustic energy;
sensing a first physiological characteristic associated with said first subject;
sensing a second physiological characteristic associated with a second subject;
determining at least one gastrointestinal parameter associated with said first subject as a function of said first acoustic energy signal.

40. The method of claim 39, wherein said second subject comprises a fetus of said first subject.
